(19) United States
(12) Patent Application Publication
(10) Pub. No.: US 2003/0157026 A1
(43) Pub. Date: Aug. 21, 2003

(54) Photodynamic diagnosis and therapy for insulin-dependent diabetes mellitus

(76) Inventors: Katsuo Aizawa, Tokyo (JP); Masao Kanazawa, Tokyo (JP); Keiji Saito, Kawasaki-shi (JP); Satoshi Imai, Kawasaki-shi (JP)

Correspondence Address: VARNDELL & VARNDELL, PLLC, 106-A S. COLUMBUS ST., ALEXANDRIA, VA, 22314, US

(21) Appl. No.: 10/395,235

(22) Filed: Mar. 25, 2003

Related U.S. Application Data
(63) Continuation-in-part of application No. PCT/JP02/06510, filed on Jun. 27, 2002.
(60) Provisional application No. 60/367,487, filed on Mar. 27, 2002.

(30) Foreign Application Priority Data
Jun. 27, 2001 (JP) 2001-194999

(51) Int. Cl. A61K 49/00

(52) U.S. Cl. 424/9.61

(57) ABSTRACT

This invention relates to a method for diagnosing insulin-dependent diabetes mellitus, which comprises administering to a mammal such a photosensitizer or an X-ray absorbing substance that has a property capable of accumulating specifically in the macrophages having infiltrated the inflammatory sites of Langerhans islets in the pancreas and also has a property capable of being activated upon absorption of a light ray or an X-ray of a specific wavelength, then irradiating the pancreas with the light ray or X-ray of the specific wavelength, and detecting or judging whether the fluorescence of the photo-excited photosensitizer or the X-ray absorption image occurs in the inflammatory sites of Langerhans islets or not.

This invention also relates to a method for treating therapeutically insulin-dependent diabetes mellitus, which comprises administering to a mammal with the diabetes mellitus such a photosensitizer or an X-ray absorbing substance that has a property capable of accumulating specifically in the macrophages having infiltrated the inflammatory sites of Langerhans islets in the pancreas and also has a property capable of being activated upon absorption of a light ray or an X-ray of a specific wavelength so as to damage or destroy the macrophages, and then irradiating the inflammatory sites of Langerhans islets with the light ray or X-ray of the specific wavelength at an intensity and for a time sufficient to damage or destroy the macrophages having infiltrated the Langerhans islets.

### CROSS REFERENCE TO RELATED APPLICATIONS

This application is a continuation-in-part application of International PCT application No. PCT/JP02/06510, filed on Jun. 27, 2002; and is the non-provisional application of prior provisional application Serial No. 60/367,487, filed on Mar. 27, 2002.

### BACKGROUND OF THE INVENTION

1. Field of the Invention

This invention relates to a photodynamic diagnostic method and a photodynamic therapeutic method for insulin-dependent diabetes mellitus (IDDM).

2. Description of Related Art

Diabetes mellitus is a disease of which major characteristic is a chronic hyperglycemia attributable to a deficiency of the action of insulin in the body of a patient and which is accompanied by a variety of characteristic metabolic disorders. Diabetes mellitus is classified into insulin-dependent diabetes mellitus and insulin-independent diabetes mellitus.

Insulin-dependent diabetes mellitus (abbreviated as IDDM), which occurs most in the ages of infancy or youth, is diabetes mellitus which can be diagnosed before the age 30-year-old. IDDM accounts for 10 to 15% of the whole patients of diabetes mellitus. The symptom of IDDM occurs abruptly, and IDDM advances to a condition called diabetic ketoacidosis.

IDDM occurs as a result of a selective destruction of the insulin-secreting Langerhans islet β cells in the pancreas as induced at a high degree of 90% or more by autoimmune reactions. In order to succeed in treating the autoimmune reactions involved in IDDM, it is necessary to initiate the therapy at the earliest stage of IDDM and to supply exogenous insulin from the outside to a patient who has been deprived of the ability to produce insulin in vivo. To make the supply of exogenous insulin unnecessary, the progress of the disease should be stopped within the period when the patient has retained a number of pancreatic β cells enough to produce a sufficient amount of endogenous insulin.

Diagnosis of IDDM is carried out mainly by judging whether an antibody ICA (namely, Islet Cell Antibody) against pancreatic β cells, for example, an anti-GAD (namely, glutamic acid decarboxylase) antibody, is positive or not, and additionally by examining serum C-peptide reaction through such a glucagon loading test which is a method of evaluating an ability to secrete endogenous insulin, with reference to the clinical progress of the patient such as mode of the onset and the presence or absence of ketosis, as well as examination of HLA type [refer to Morbid State of Type I Diabetes Mellitus and its Guideline for Diagnosis: Mebio 2, pp. 28-38 (2000)]. However, diagnosis in all of these examination items is not yet determinative as an indicator for examination of detecting the destruction of pancreatic β cells.

Further, the insulin therapy may be effective in treatment of IDDM, but insulin should be injected regularly every day to keep the life of the patient. The creation of a physiological pattern of spontaneous insulin secretion of patient which is attained by administering exogenous insulin is considered to bring about good control of the blood-sugar level, and an enhanced insulin therapy comprising a continuous subcutaneous insulin infusion II (CS II) is actively used.

However, a time of about 2 hours is required for an administered prompt insulin preparation to reach the peak of its action, and a time of about 6 to 8 hours passes before the action of insulin disappears. Accordingly, there often occurs the case where, during the insulin therapy, a phenomenon such as hypoglycemia appears in 3 to 4 hours after a meal. Further, even after the insulin therapy has been initiated, ketoacidosis may occur upon a neglect of the insulin injection or under the action of stresses as induced by infectious diseases, accidents or severe medical treatment. Further, the insulin therapy is a symptomatic therapy and IDDM occurs in persons of young age, so that the injection of insulin is required every day for a long period of time, which gives a heavy burden on the patients. Accordingly, there is now still a demand for a new therapeutic method, which can be substituted for the insulin therapy.

As described above, IDDM onsets as a result of the selective destruction of the insulin-secreting pancreatic cells as induced at a high degree of 90% or more. Recently, it was reported that, just after the onset of IDDM, about 30% of the pancreatic β cells can remain survived without being destroyed, and also that, in an animal model just before the onset of IDDM, the total volume of the pancreatic β cells can maintain their original volume [refer to Shimada et al, Diabetes, 45, 1063-1067 (1996)]. Further, it is also reported that human patients of anti-GAD-antibody-positive diabetics who have an ameliorated ketosis by a dietary cure, or human patients of autoantibody-positive diabetics who have developed diabetes mellitus by ketoacidosis, can become needless to be supplemented with exogenous insulin because of the disappearance of autoantibody [refer to Morimoto et al, Diabetes Care, 21(11), pp. 2037-2039 (1998)].

From these reports, it can be estimated that a large number of the surviving pancreatic β cells remain still at an initial stage of the onset of IDDM. Accordingly, if early diagnosis of IDDM can be made feasible and if the autoimmune reaction causative for IDDM can be terminated at an early stage of the onset of IDDM, the destruction of pancreatic β cells can be minimized, while the Langerhans islets can still secure their ability to produce endogenous insulin. In addition, if early treatment of IDDM can be accomplished, the patient can be set free from the long term injection of insulin.

For IDDM, there are fortunately many kinds of useful animal models such as BB rats and NOD mice, and such animal models can be used to study the mechanism of the onset of IDDM as well as therapeutic methods for IDDM (refer to Rossini et al, Ann. Rev. Immunol., 3, pp. 289-320 (1985)).

It is known that in particular, BB rats, either male or female, can develop IDDM on Day 60 to 120 after birth, and IDDM as developed in the BB rats is similar in many respects to human IDDM. Thus, the BB rats develop typical IDDM due to a known cause that, based on their hereditary background, pancreatic insulitis; namely an inflammation of the Langerhans islets occurs via the mechanism of the autoimmune reactions, which leads to the destruction of the β cells in the inflammatory sites of Langerhans islets. It is found that the immune cells having infiltrated the inflammatory sites of Langerhans islets in the pancreatic insulitis upon an onset of IDDM in BB rats comprise macrophages, T lymphocytes, NK cells, neutrophils, eosinophils and others. It is thought that at first, macrophages begin to infiltrate the inflammatory sites of Langerhans islets and then T lymphocytes and NK cells infiltrate the inflammatory sites to participate in the destruction of the β cells (refer to Like AA et al., Springer-Verlag 269-284 (1988) and Like AA et al., J. Exp. Med. 164, pp. 1145 (1986)). It is also known that, in human IDDM, the infiltration of macrophages into the inflammatory sites of Langerhans islets occurs in the pancreas of a human.

### SUMMARY OF THE INVENTION

If the macrophages having infiltrated the inflammatory sites of Langerhans islets could be selectively damaged or destroyed at an early stage of the onset of IDDM so that the pancreatic β cells could be prevented from being destroyed by the infiltrating macrophages, to protect the pancreatic β cells to a certain degree, it is then expectable that the onset of human IDDM could be controlled.

An object of this invention is to provide a method capable of making an early diagnosis of insulin-dependent diabetes mellitus (IDDM) and a method capable of making an early therapeutic treatment of IDDM. Another object of this invention is to provide a diagnostic composition which can be used effectively in a photodynamic diagnosis of IDDM, as well as a therapeutic composition which is effective in a photodynamic therapeutic treatment of IDDM.

We, the present inventors, have conducted investigations in order to achieve the objects described above, and as a result, the present inventors have now found for the first time that a certain kind of photosensitizer has a property capable of being specifically and selectively uptaken into the macrophages having infiltrated the inflammatory sites of Langerhans islets in the pancreas of a mammal with IDDM as well as a property capable of being activated upon absorption of a light ray of a specific wavelength to emit a fluorescence. Further, the present inventors have now found that, when the inflammatory sites of Langerhans islets containing therein the macrophages, where a photosensitizer having the aforesaid properties has been accumulated, are irradiated with a selected light ray of a specific wavelength, a photodynamic diagnosis and a therapeutic treatment of the inflammatory sites of Langerhans islets are possible to be conducted. Thus, it has now been found that, when there is conducted a method which comprises administering to a mammal with IDDM a pharmaceutical composition containing a photosensitizer having the aforesaid properties as an active ingredient; allowing to lapse a time sufficient for the administered photosensitizer having the aforesaid properties to be uptaken into the macrophages having infiltrated the inflammatory sites of Langerhans islets in the pancreas of the mammal with IDDM; and after the elapse of such time, irradiating the inflammatory sites of Langerhans islets where the infiltrating macrophages having uptaken the administered photosensitizer therein are present, with a selected light ray of a specific wavelength suitable for photo-excitation of the administered photosensitizer, then the photosensitizer as uptaken in said macrophages can emit a fluorescence unique to said photosensitizer, and the inflammatory sites of Langerhans islets containing the infiltrating macrophages can be visible brightly due to the emitting fluorescence. Accordingly, it has now been found at this time that the onset of IDDM can be diagnosed through a photodynamic therapy (abbreviated as PDT) by detecting the fluorescence-emitting inflammatory sites of Langerhans islets by means of an endoscope.

The present inventors have now recognized that a known compound used as a photosensitizer in conventional methods of PDT, which is talaporfin, namely mono-L-aspartyl chlorin e6 represented by the following formula (I):

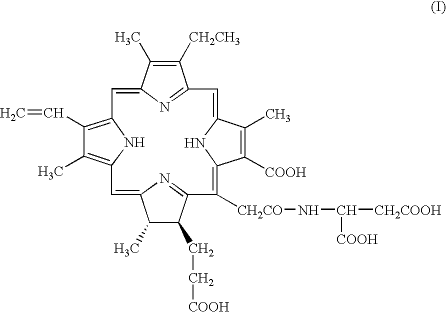

or a pharmaceutically acceptable salt or a solvate thereof, is very excellent and effective for use in the method of photodynamic therapy.

Further, the present inventors have now found that, when various photosensitizers utilized generally as photosensitive agents in conventional methods of PDT, for example, known chlorin e6, chlorin e6 derivatives, benzoporphyrin derivatives, rostaporfin, motexafin lutetium, ATX-S10, temoporfin (Foscan; trade name), and porfimer sodium (Photofrin; trade name) (refer to e.g., JP patent publication 6-88902, JP patent publication 6-89000, and U.S. Pat. Nos. 4,675,338, 6,162,242, 5,095,030 and5,756,541) are each singly administered, these photosensitizers have a property such that they can selectively be uptaken by and into the macrophages having infiltrated the inflammatory sites of Langerhans islets in the pancreas of a mammal with IDDM.

Further, the present inventors have now found that, when there is administered an X-ray absorbing substance which has a property such that, upon irradiation with an X-ray of a wavelength of 0.153 Å, it can absorb the X-ray of such wavelength, and which is namely a mono-L-aspartyl chlorin e6-gold complex represented by the following formula (II),

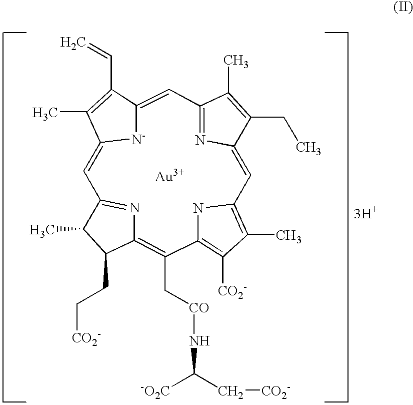

or a pharmaceutically acceptable salt thereof or a solvate thereof (refer to PCT Application International Publication No. WO 00/02882), said gold complex can exhibit a property such that it can be uptaken in the macrophages having infiltrated the inflammatory sites of Langerhans islets in the pancreas of a mammal with IDDM.

When an X-ray of a wavelength of 0.153 Å is irradiated from an X-ray source placed in the outside of the body selectively to the pancreas, the gold complex of formula (II), which has been accumulated in the macrophages having infiltrated the inflammatory sites of Langerhans islets in the pancreas and also having contained the uptaken gold complex of formula (II), is able to absorbs the X-ray, and thus an image of the X-ray absorption in the inflammatory sites of Langerhans islets can be detected by means of X-ray radiography. Accordingly, the presence or absence of the inflammatory sites of Langerhans islets can be detected and diagnosed by a method which comprises administering the gold complex of formula (II), and subsequently conducting an X-ray radiography.

On the basis of the inventors' findings described above, this invention has been accomplished.

Accordingly, in a first aspect of this invention, there is provided a method for diagnosing insulin-dependent diabetes mellitus, which comprises:

administering to a mammal a sufficient amount of a photosensitizer for accumulating the photosensitizer in the macrophages having infiltrated the inflammatory sites of Langerhans islets in a pancreas of the mammal, the photosensitizer being capable of accumulating specifically in the macrophages having infiltrated the inflammatory sites of Langerhans islets in the pancreas of the mammal and being activated upon absorption of a light ray of a specific wavelength;

irradiating the pancreas with the light ray of the specific wavelength; and

detecting fluorescence of the photo-excited photosensitizer in the inflammatory sites of Langerhans islets.

Further, in a second aspect of the invention, there is provided a diagnostic composition for insulin-dependent diabetes mellitus, which comprises as an active ingredient a photosensitizer having a property that it can be uptaken specifically and selectively in the macrophages having infiltrated the inflammatory sites of Langerhans islets in the pancreas of a mammal with insulin-dependent diabetes mellitus, as well as a property that it can be activated upon its absorption of a light ray of a specific wavelength to emit a fluorescence.

In the diagnostic method for insulin-dependent diabetes mellitus according to the first aspect of the invention, the photosensitizer to be administered may be chlorin e6, a chlorin e6 derivative, a benzoporphyrin derivative, rostaporfin, motexafin lutetium, ATX-S10, temoporfin or porfimer sodium, or a pharmaceutically acceptable salt thereof.

The photosensitizer to be given as an effective agent in the diagnostic method for IDDM according to the first aspect of the invention may preferably be mono-L-aspartyl chlorin e6 represented by formula (I):

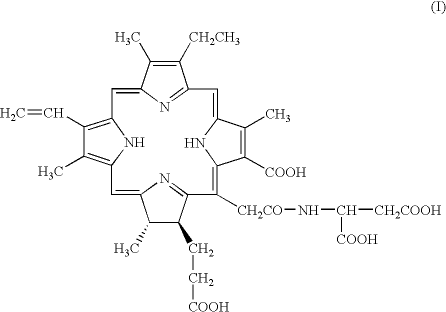

or a pharmaceutically acceptable salt or a solvate thereof.

The diagnostic composition for insulin-dependent diabetes mellitus (IDDM) according to the second aspect of the invention can also be used in such a method for photodynamic diagnosis of insulin-dependent diabetes mellitus, wherein the composition containing said photosensitizer is administered intravenously, intramuscularly or subcutaneously or via a pancreatic duct to a mammalian subject with the diabetes mellitus, and wherein, after there is allowed an elapse of such a sufficient time during which the administered photosensitizer has been uptaken in the macrophages present in the Langerhans islets in the pancreas of the mammalian subject, at least a part of the pancreas is then irradiated with a light ray of a specific wavelength suitable for photo-excitation of the administered photosensitizer, and subsequently there is detected or judged the presence or absence of a fluorescence as emitted from the macrophages which have infiltrated the inflammatory sites of Langerhans islets and which have uptaken therein selectively the photosensitizer.

Furthermore, in a third aspect of this invention, there is provided a method for diagnosing insulin-dependent diabetes mellitus, which comprises:

administering to a mammal a sufficient amount of an X-ray absorbing substance for accumulating the X-ray absorbing substance in the macrophages having infiltrated the inflammatory sites of Langerhans islets in a pancreas of the mammal, the X-ray absorbing substance being capable of accumulating specifically in the macrophages having infiltrated the inflammatory sites of Langerhans islets in the pancreas and being activated upon absorption of an X-ray of a specific wavelength;

irradiating the pancreas with the X-ray of the specific wavelength; and

detecting an X-ray absorption image in the inflammatory sites of Langerhans islets.

Further, in a fourth aspect of the invention, there is provided a diagnostic composition for insulin-dependent diabetes mellitus, which comprises as an active ingredient an X-ray absorbing substance that has a property capable of accumulating specifically in the macrophages having infiltrated the inflammatory sites of Langerhans islets in the pancreas of a mammal with IDDM.

In the diagnostic method of the third aspect of the invention, the X-ray absorbing substance to be administered may preferably be a mono-L-aspartyl chlorin e6-gold complex represented by formula (II):

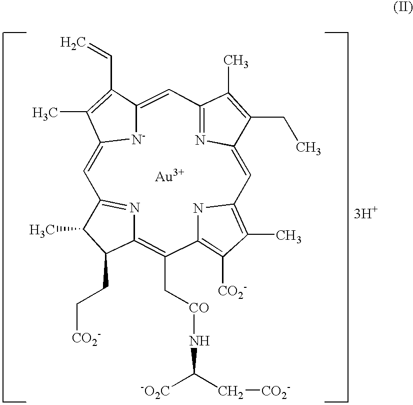

or a pharmaceutically acceptable salt or a solvate thereof.

The diagnostic composition for IDDM according to the fourth aspect of the invention can be used also in such a method for photodynamic diagnosis of insulin-dependent diabetes mellitus, wherein the composition containing said X-ray absorbing substance is administered intravenously, intramuscularly or subcutaneously or via a pancreatic duct into a mammal with the diabetes mellitus, and wherein, after there is interposed an elapse of such a sufficient time during which the administered X-ray absorbing substance has been uptaken in the macrophages having infiltrated the Langerhans islets of the pancreas of the mammal, the pancreas is irradiated with an X-ray beam containing an X-ray of a specific wavelength as emitted from an X-ray generator placed outside the body of the mammal, and the X-ray beam having passed through the body of the mammalian subject is photographed on an X-ray film, and with the resulting X-ray absorption image as photographed on the X-ray film, there is detected or judged the presence or absence of an X-ray absorption image attributable to a group of the macrophages which have infiltrated the inflammatory sites of Langerhans islets in the pancreas and which have uptaken therein selectively the administered X-ray absorbing substance.

When the photosensitizer used as an effective agent in the diagnostic method of the first aspect of the invention, for example, the compound of formula (I) above, is administered into a human or a mammal with IDDM, the administered photosensitizer is uptaken selectively into the macrophages present in the inflammatory sites of Langerhans islets, that is, the IDDM lesion sites. The administered photosensitizer, upon being irradiated with a light ray having a wavelength suitable for photo-excitation of the photosensitizer, can absorb the light ray to emit a fluorescence, and when the intensity of irradiation of the light ray is then increased, the photosensitizer as uptaken and accumulated in the macrophages is activated by absorbing the light ray, to generate active oxygen and thereby exhibit a cell-killing effect on the macrophages, whereby the macrophages can be damaged or destroyed. In particular, the compound of formula (I), upon the irradiation with a red laser light containing a light of a wavelength of 664 nm, can be activated by absorbing the 664 nm light ray, to generate active oxygen and thereby exhibit a cell-killing effect on the macrophages.

When the X-ray absorbing substance, for example, the compound of formula (II), which is used as an effective agent of the diagnostic method in the third aspect of the invention, is irradiated with an X-ray beam containing an X-ray of a specific wavelength, for example a wavelength of 0.153 Å, it is activated by absorbing the X-ray, and it can exhibit a cell-killing effect by irradiation with the X-ray. Upon irradiation with the X-ray beam, the macrophages containing the X-ray-absorbing substance therein can be damaged or destroyed.

Accordingly, the photosensitizer used as the effective agent in the first aspect method of the invention as well as the X-ray absorbing substance used in the third aspect method of the invention, when it has been uptaken in the macrophages and is then irradiated with a light ray or an X-ray at an irradiance sufficient to exert its cell-killing effect, can damage or destroy the macrophages having infiltrated the inflammatory sites of Langerhans islets, whereby the inflammatory sites of Langerhans islets can be treated therapeutically, enabling a photodynamic therapy (PDT) for IDDM to be made feasible.

Accordingly, in a fifth aspect of the invention, there is provided a method for treating therapeutically insulin-dependent diabetes mellitus, which comprises:

administering to a mammal with the diabetes mellitus a sufficient amount of a photosensitizer for accumulating the photosensitizer in the macrophages having infiltrated the inflammatory sites of Langerhans islets in a pancreas of the mammal, the photosensitizer being capable of accumulating specifically in the macrophages having infiltrated the inflammatory sites of Langerhans islets in the pancreas and being capable of generating, upon absorption of a light ray of a specific wavelength, active oxygen for damaging or destroying the macrophages; and

irradiating the inflammatory sites of Langerhans islets with the light ray of the specific wavelength at an irradiance and for a time sufficient to damage or destroy the macrophages having infiltrated the Langerhans islets.

Further, in a sixth aspect of this invention, there is provided a therapeutic composition for insulin-dependent diabetes mellitus, comprising as an active ingredient a photosensitizer that has a property of being specifically and selectively uptaken into the macrophages having infiltrated the inflammatory sites of Langerhans islets in the pancreas and also has a property of being activated upon absorption of a light ray of a specific wavelength to generate active oxygen and damage or destroy the macrophages.

The photosensitizer used as an effective agent in the therapeutic method in the fifth aspect of the invention may be chlorin e6, a chlorin e6 derivative, a benzoporphyrin derivative, rostaporfin, motexafin lutetium, ATX-S10, temoporfin or porfimer sodium.

The photosensitizer to be administered as an effective agent in the therapeutic method of the fifth aspect of the invention may preferably be mono-L-aspartyl chlorin e6 of formula (I) above, or a pharmaceutically acceptable salt thereof or a solvate thereof, and the light ray to be irradiated in this case is red laser lights containing a light of a wavelength of 664 nm.

The therapeutic composition for IDDM according to the sixth aspect of the invention can be used, for example, also in such a method of photodynamic therapy for insulin-dependent diabetes mellitus, which comprises administering the composition containing said photosensitizer intravenously, intramuscularly or subcutaneously or via a pancreatic duct to a mammal with insulin-dependent diabetes mellitus, allowing an elapse of a time enough for the administered photosensitizer to be uptaken in the macrophages present in the Langerhans islets of the pancreas of the mammal, and irradiating the inflammatory sites of Langerhans islets in the pancreas with a light ray of a specific wavelength suitable for photo-excitation of the administered photosensitizer, in such way that the light irradiance is so regulated that the photosensitizer as uptaken in the macrophages present in the Langerhans islets is activated to generate active oxygen and damage or destroy the macrophages, whereby the macrophages having infiltrated the Langerhans islets are damaged or destroyed by the irradiation of the light rays.

In a seventh aspect of the invention, there is provided a method for treating therapeutically insulin-dependent diabetes mellitus, which comprises:

administering to a mammal with the diabetes mellitus a sufficient amount of an X-ray absorbing substance for accumulating the X-ray absorbing substance in the macrophages having infiltrated the inflammatory sites of Langerhans islets in a pancreas of the mammal, the X-ray absorbing substance being capable of accumulating specifically in the macrophages having infiltrated the inflammatory sites of Langerhans islets in the pancreas and being activated upon absorption of an X-ray of a specific wavelength; and

irradiating the pancreas with the X-ray of the specific wavelength by irradiation of a beam of X-rays at an intensity and for a time sufficient to damage or destroy the macrophages having infiltrated the Langerhans islets.

In an eighth aspect of this invention, there is further provided a therapeutic composition for treating insulin-dependent diabetes mellitus, comprising as an active ingredient an X-ray absorbing substance that has a property of being specifically and selectively uptaken in the macrophages having infiltrated the inflammatory sites of Langerhans islets and also has a property by which said substance absorbs X rays and damages or destroys said macrophages by irradiation with X-rays.

The X-ray absorbing substance used as an effective agent in the therapeutic method of the seventh aspect of the invention may preferably be the mono-L-aspartyl chlorin e6-gold complex of formula (II) or a pharmaceutically acceptable salt or a solvate thereof.

The therapeutic composition according to the eighth aspect of the invention can be used, for example, also in such a method of photodynamic therapy for insulin-dependent diabetes mellitus, which comprises administering the composition containing said X-ray absorbing substance intravenously, intramuscularly or subcutaneously or via a pancreatic duct to a mammal with insulin-dependent diabetes mellitus, allowing an elapse of a time enough for the administered X-absorbing substance to be uptaken in the macrophages present in the islets of Langerhans of the pancreas of the mammal, and then irradiating selectively the pancreas of the mammal with an X-ray beam, for example, X-ray beam containing an X-ray having a wavelength of 0.153 Å, as emitted from an X-ray generator placed outside the body of the mammal, in such way that the irradiance of X-ray applied is so regulated that the X-ray absorbing substance as uptaken and contained in the macrophages present in the islets of Langerhans is activated by absorption of the X-ray to damage or destroy the macrophages, whereby the macrophages having infiltrated the islets of Langerhans can be damaged or destroyed by irradiation of the X-ray beam.

In the diagnostic compositions according to the second and fourth aspects of the invention and also in the therapeutic compositions according to the sixth and eighth aspects of the invention, the compound to be used as the active ingredient may be mixed with a pharmaceutically acceptable carrier. For example, the composition can be in the form of a solution in a suitable liquid carrier, for example, physiological saline, a glucose solution in water, and others. The composition in this solution form can be administered by a suitable method into the body of a patient to be diagnosed or treated. The composition can be formulated not only into an aqueous solution but also into an aqueous suspension having the active ingredient dispersed in water with aid of a suitable dispersant. Administration may be conducted preferably by direct injection into a blood vessel, but may be conducted also by muscular or subcutaneous administration. Further, topical administration via a pancreatic duct can be conducted using a catheter.

The compositions according to the second, fourth, sixth and eighth aspects of the invention can contain a known binder, a pH adjusting agent such as phosphoric acid, hydrochloric acid or sodium hydroxide, an excipient, a preservatives such as para-hydroxybenzoic acid derivatives, a solvent such as water, ethanol or glycol, and an isotonizing agent such as sugar or sodium chloride. These compositions according to the second, fourth, sixth and eighth aspects of the invention can contain, for example, the compound of formula (I) or the compound of formula (II) in the form of its salt with a base, for example, a sodium salt, and can be formulated into a sterile, pyrogen-free lyophilized preparation.

The photosensitizer or the X-ray absorbing substance to be used as an effective agent in the photodynamic diagnostic methods for IDDM in the first and third aspects of the invention, as well as in the photodynamic therapeutic methods in the fifth and seventh aspects of the invention is preferred to have the following characteristics:

(a) When it is not activated with light ray or X-rays or until it is activated with light rays or X-rays, it shall be nontoxic to the host to whom it is given at a dose necessary for the diagnosis and therapy.

(b) It can be uptaken selectively into the macrophages having infiltrated in the inflammatory sites of Langerhans islets in the pancreas with IDDM.

(c) It is selectively activated upon irradiation with a light ray of a specific wavelength, for example, a light ray such as visible light, electromagnetic waves, X-rays, etc.

(d) Upon being irradiated with a light ray of a specific wavelength, it can generate a specific and measurable fluorescence or it can absorb X-rays.

(e) Upon irradiation of a light ray or an X-rays of a specific wavelength, it can selectively damage or kill the macrophages having infiltrated the inflammatory lesion sites of Langerhans islets without affecting the surrounding normal tissue regions of the human body.

(f) After IDDM therapy was done, it can be easily metabolized in the body or excreted out of the body of patient.

As the photosensitizer or X-ray absorbing substance having all the characteristics described above, the compound of formula (I) and the compound of formula (II) given hereinbefore can be mentioned. The compound of formula (I) and the compound of formula (II), which are used preferably according to this invention, have the characteristics described above, and are advantageous in that they can be dissolved at a suitable solubility in a water having a physiological hydrogen ion concentration (pH). Further, it has now been found that the compound of formula (I) can generate a fluorescence of higher intensity than by the same dosage of other photosensitizers. Accordingly, when the compound of formula (I) or the compound of formula (II) is administered in the methods of this invention or used as the active ingredient in the compositions of this invention, that compound can be distributed stably and uniformly at a higher concentration in the inflammatory sites of pancreatic β cells than in the normal tissues surrounding the lesion site, and accordingly, it is possible to obtain a fluorescence image or an X-ray image having more significant contrast. In particular, the compound of formula (I) can be excited by irradiation with red laser lights containing a light of a wavelength of 664 nm, so as to emit a fluorescence, and upon its transition to the ground state, it generates active oxygen, thereby exerting the cell-killing effect on the macrophages.

In particular, the compound of formula (II) has a property to show Auger effect such that the compound (II) having received the irradiation of X-ray beam containing an X-ray of a wavelength of 0.153 Å is excited by absorption of the X ray of said wavelength, and upon its transition to the ground state, the compound of formula (II) gives energy to other electrons within the gold atoms to release the electrons, thereby exhibiting the cell-killing effect on the macrophages.

When the compound of formula (I) above or the compound of formula (II) above is administered in the photodynamic therapeutic methods for IDDM according to this invention with using the composition of the invention, the dose of the compound administered may vary depending on the method of administration or the object to be achieved. In the case of systemic administration via a vein, the compound of formula (I) or the compound of formula (II) may be administered at a dose of 0.1 mg/kg to 5 mg/kg, more preferably 0.2 mg/kg to 2 mg/kg. In the case of topical administration, it is appropriate to administer at a proper dosage such compound that can afford a fluorescence image or an X-ray absorption image of the Langerhans islet disordered sites in the pancreas having IDDM, which is observable in a high contrast. For example, it is suitable to use a method of injecting, via a pancreatic duct, few ml of a solution which is prepared by dissolving the compound of formula (I) or the compound of formula (II) at a concentration of 0.1 to 1 mg/ml in a volume of physiological saline.

The compound of formula (I) and the compound of formula (II) are evidently non-toxic as long as it is administered at a dose usually used in the diagnosis or therapy according to this invention. For example, even when talaporfin, namely the compound of formula (I) was administered at a dose of up to 20 mg/kg, none of significantly toxic observations was found in experimental animals. In addition, a benzoporphyrin derivative such as verteporfin, rostaporfin (Purlytin; trade name), motexafin lutetium, ATX-S10, temoporfin (Foscan; trade name) or porfimer sodium (Photofrin) is suitable as the photosensitizer to be used in the diagnostic method of the first aspect of the invention or in the therapeutic method of the fifth aspect of the invention. These photosensitizers can be used in a free form or in the form of a pharmaceutically acceptable salt thereof or a solvate thereof as the effective agent in the methods of the invention. The benzoporphyrin derivative is administered at a dose of 0.1 to 0.5 mg/kg and may be irradiated with a laser light of a wavelength of 690 nm. Rostaporfin is administered at a dose of 0.2 to 1 mg/kg and may be irradiated with a laser light of a wavelength of 665 nm; motexafin lutetium is administered at a dose of 1 to 3 mg/kg and may be irradiated with a laser light of a wavelength of about 730 nm; ATX-S10 or temoporfin is administered at a dose of 0.1 to 0.5 mg/kg and may be irradiated with a laser light of a wavelength of 652 nm; and porfimer sodium is administered at a dose of 1 to 3 mg/kg and may be irradiated with a laser light of a wavelength of 630 nm. When the photosensitizer or a composition of the invention comprising the photosensitizer, for example, the compound of formula (I) as the active ingredient is administered, the compound of formula (I) after the administration thereof can be uptaken specifically and selectively into the macrophages having clustered in the lesion sites of IDDM where the macrophages acting on the pancreatic β cell have infiltrated, resulting in that the compound of formula (I) can accumulate in the inflammatory sites. After there is allowed an elapse of a suitable time from the administration of the compound, for instance, after there is allowed a time elapse of few minutes to 48 hours, preferably few minutes to 24 hours in the case of intravascular administration of the compound of formula (I) so as to make an elapse of such a time which is sufficient to permit the compound of formula (I) to be uptaken selectively into the macrophages having clustered in the inflammatory sites of Langerhans islets, the inflammatory sites of Langerhans islets are then irradiated with a light ray of a specific wavelength, preferably a laser light via an optical fiber which has been inserted via the skin and via a pancreatic duct into said inflammatory sites or into a blood vessel near to said inflammatory sites. The irradiation of the light ray to be applied to the compound of formula (I) should necessarily be made at such an irradiance as to permit visible observation of a fluorescence image. In the case of the compound of formula (I), red laser lights containing a light of a wavelength of 664 nm are applied usually at an irradiance of 20 to 200 J/cm².

Further, when an X-ray absorbing substance such as the compound of formula (II) is administered, it is necessary that the X-rays to be irradiated should be applied at such an irradiance that the X-ray beam having passed through the body of patient can be observed as an X-ray absorption image on an X-ray film or in another X-ray photography unit. When the compound of formula (II) is administered, X-ray beam containing an X-ray of a wavelength of 0.153 Å is applied at an energy of 5 to 50 keV.

When endoscopic retrograde cholangio-pancreatography (ERCP) can be used for irradiation of the inflammatory sites of Langerhans islets with light rays, an ERCP unit is used for the irradiation of laser lights. The inflammatory sites of Langerhans islets with IDDM as irradiated with light rays can emit a fluorescence selectively, and thus the IDDM lesion sites which are emitting this fluorescence can be observed directly with naked eyes by means of an inserted optical fiber scope or on a CRT screen scope.

For irradiating the inflammatory sites of Langerhans islets with laser lights, the photosensitizer is administered as an effective agent, and then the inflammatory sites of Langerhans islets are irradiated with laser lights as emitted from the top of a quartz fiber which has been inserted in the vicinity of the inflammatory sites of Langerhans islets. Because the pancreas is close to the pylorus in a lower part of the stomach and close to the duodenum, the pancreas can be irradiated with laser lights as emitted from such an endoscope which is equipped with a laser light-irradiating fiber and which has been inserted into the gastric cavity or duodenal cavity.

On the other hand, when the X-ray absorbing compound of formula (II) is administered, an X-ray beam containing an X-ray of a wavelength of 0.153 Å is to be applied as described above, and the X-ray beam containing the X-ray of a wavelength of 0.153 Å is available by using a known X-ray generator. However, when a coherent X-ray is to be used, a large-scale radiation facility, Spring-8, in Harima Science Garden City, Japan or radiation facilities in High Energy Accelerator Research Organization in Tsukuba City, Japan can be utilized.

After the X-ray absorbing substance, for example the compound of formula (II), has been uptaken into the infiltrating macrophages after the administration of e.g., the compound of formula (II) to the body of a mammal with IDDM, by allowing an elapse of a time of few minutes to 48 hours, preferably few minutes to 24 hours from the administration, the inflammatory sites of Langerhans islets and the surrounding region are then irradiated with X-ray beam. The X-ray beam is applied usually from the outside of the body. The X-ray having passed through the pancreas in the body of the mammal is received on an X-ray film to form an X-ray absorption image thereon, whereby the inflammatory sites can be observed as an X-ray absorption image. With the resultant X-ray absorption image, IDDM can be diagnosed. Further, when the irradiance of X-ray is increased, the excited X-ray absorbing substance is able to damage or destroy the infiltrating macrophages having uptaken the X-ray absorbing substance therein, by the action of the X-ray irradiation or by the Auger effect in the case of administration of the compound of formula (II).

In an IDDM therapy with using the therapeutic method according to the fifth or seventh aspect of the invention, the present inventors have now devised two procedures, one is a therapeutic process of making the irradiation of laser lights emitted from a fiber catheter as inserted into the inflammatory sites of Langerhans islets, and the other is a therapeutic process of making the irradiating of X-ray beam from the outside of the body. In the first procedure involving the insertion of a fiber catheter into the inflammatory sites of Langerhans islets, the pancreatic β cells are directly irradiated with laser lights, and a fluorescence unique to the administered photosensitizer is observed, whereby the positions of the inflammatory sites of Langerhans islets are decided accurately, and thus a direct therapy of the inflammatory sites can be conducted accurately and topically. In the second procedure of making the irradiation of X-rays, the inflammatory sites of Langerhans islets are irradiated with X-ray from the outside of the body and an X-ray image of the pancreas is observed, whereby the positions of the Langerhans islet lesion sites can be grasped, and so a therapy of them can be conducted directly, accurately and topically in a non-invasive manner.

### BRIEF DESCRIPTION OF THE DRAWING

FIG. 1 is a graph which shows a curve of plotting the survival rate (%) of a therapeutically treated group of BB rats having received a therapeutic method comprising the administration of mono-L-aspartyl chlorin e6 tetrasodium salt and the irradiation of a laser light after the onset of IDDM in the BB rats in Test Example 3 given hereinafter, in comparison with a curve of plotting the survival rate (%) of the control group (untreated) of the BB rats having the onset of IDDM.

### DESCRIPTION OF EMBODIMENT OF THE INVENTION

Hereinafter, this invention is illustrated in more detail with reference to Test Examples below, but this invention is not limited to these Examples.

### TEST EXAMPLE 1

BB rats, which had exhibited a morbid state of IDDM morphologically and biochemically similar to human IDDM, were used as test animals. From 60 days after birth, the BB rats were examined every day for the urine sugar level and blood sugar level by using GLUTEST SENSOR (Sanwa Kagaku Co., Ltd., Japan). Those BB rats whose urine sugar became positive to confirm the onset of IDDM were administered with tetrasodium salt of mono-L-aspartyl chlorin e6 of formula (I) at a dose of 5 mg/kg via a tail vein. One hour after the administration, the abdomens of the rats were opened under ether anesthesia, and their pancreases were excised. The excised pancreases were frozen in dry ice/acetone, to prepare frozen pancreatic sections of 5 μm in thickness. The frozen pancreatic sections were irradiated with a semiconductor laser light of 664 nm (Matsushita Industrial Equipment Co., Ltd., Japan). An image of fluorescence as emitted by mono-L-aspartyl chlorin e6 in the frozen pancreatic section was observed under a fluorescence microscope equipped with a notched filter for cutting 664 nm wavelength (made by Carl Zeiss). Further, the frozen pancreatic section was stained by an immunological histochemical method with rat macrophage-specific antibody (made by BMA).

As a result, the macrophage antibody-positive macrophages were found to be present in the Langerhans islets in the test pancreas section, and it was confirmed that in the BB rats, the infiltration of the Langerhans islets by the macrophages took place just after the onset of IDDM. Further, the places of the pancreas section which indicated the previously observed fluorescence of mono-L-aspartyl chlorin e6 were confirmed to be consistent with the sites where the macrophages were present in the pancreas sections. Thus, it was estimated that mono-L-aspartyl chlorin e6 as administered was uptaken into and accumulated specifically in the macrophages having infiltrated the islets of Langerhans. When a method comprising irradiation of a UV ray of a wavelength of 405 nm and observing a fluorescence at 672 nm with a cut filter for cutting light rays of wavelengths of not more than 600 nm was used for the observation of the fluorescence of mono-L-aspartyl chlorin e6, there could again be obtained the same result as that obtained in the above-described method comprising irradiation of the 664-nm semiconductor laser.

### TEST EXAMPLE 2

A sodium salt of the mono-L-aspartyl chlorin e6-gold complex of formula (II) was administered in the same manner as in Test Example 1 at a dose of 5 mg/kg via a tail vein into BB rats whose urine sugar became positive to confirm the onset of IDDM. One hour after the administration, the abdomens of the rats were opened under ether anesthesia, and their pancreases were excised. The excised pancreases were fixed with glutaric acid and fixed again and embedded in the probes for observation under an electron microscope. Thereafter, very thin test sections of the pancreas were prepared, and gold particles in the test sections could be observed in a transmitted state under an electron microscope. As a result, it was confirmed that the mono-L-aspartyl chlorin e6-gold complex had been uptaken selectively into the macrophages having infiltrated the Langerhans islets.

It was confirmed that, like mono-L-aspartyl chlorin e6, the mono-L-aspartyl chlorin e6-gold complex had also been uptaken specifically into and accumulated in the macrophages having infiltrated the Langerhans islets at an early stage of the onset of IDDM.

### TEST EXAMPLE 3

From 60 days after birth, BB rats (16 rats per group) were examined every day for the urine sugar level and blood sugar level, to confirm the time of the onset of IDDM. Those BB rats whose urine sugar became positive to confirm the onset of IDDM were administered via a tail vein with tetrasodium salt of mono-L-aspartyl chlorin e6 at a dose of 5 mg/kg. Twenty-four hours after the administration, a 664-nm laser light at an irradiance of 50 J/cm² was applied transcutaneously via the back of rats to the target sites, that is, the Langerhans islets in the pancreas and their surrounding region. Photodynamic therapy of IDDM was thus conducted once, and thereafter, the survival rate (%) of the BB rats were evaluated during the breeding for subsequent 32 days.

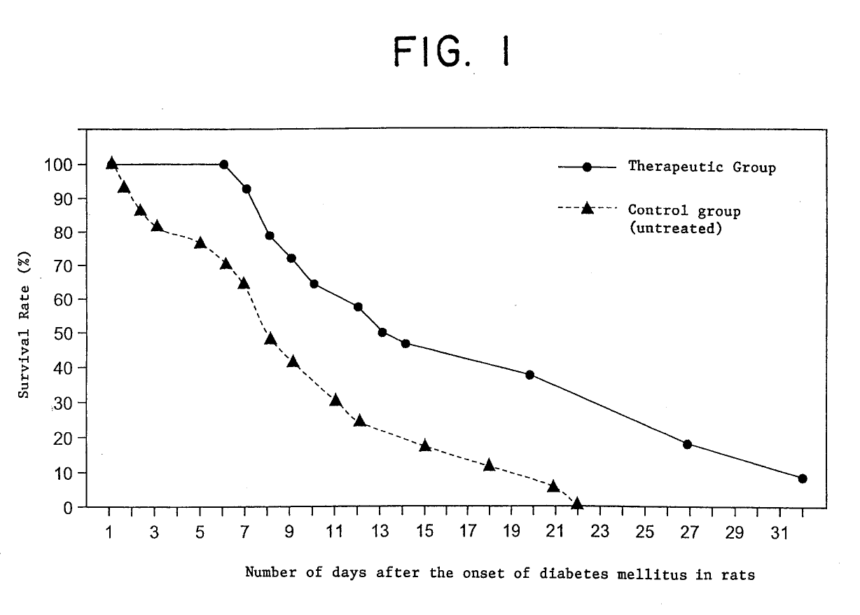

As a result, the group of rats as treated by the above photodynamic therapy showed an evident prolongation of their survival curve, as compared with that of the untreated, control group (17 rats per group). Thus, the survival rate (%) of the group of rats as treated by the photodynamic therapy was 100% on Day 6 after the start of the therapy, and several rats surviving still after Day 35 were confirmed. On the other hand, the survival rate (%) of the control group of rats (untreated) was about 65% on Day 6 after the start of the test, and all rats had died on Day 22 after the start of the test. Survival curves of showing these results (that is, the curves showing the relationship between the number of days of survival of rats and the survival rate (%) of rats under test) are shown in FIG. 1 of the attached drawing.

The survival rate (%) of rats under test was evaluated by the following equation:

$$Survival rate ( \% ) = \frac{\begin{matrix}Number of surviving rats \\ at the day of measurement\end{matrix}}{Total number of rats under test} \times 100$$

As a result of the fact that the macrophages having infiltrated the inflammatory sites of Langerhans islets were killed by the photodynamic therapy using mono-L-aspartyl chlorin e6, it was clearly demonstrated that the progress of the Langerhans islet inflammation, that is, the pancreatic insulitis was suppressed by the PDT, and the survival curve of the treated rats was improved. In the group of rats as treated by the photodynamic therapy, as compared with the control group of rats (untreated), the blood sugar level had changed at slightly lower stages, and there was observed a tendency to delay the disappearance of insulin production attributable to IDDM.

The dose of 5 mg/kg of tetrasodium salt of mono-L-aspartyl chlorin e6, with which the effectiveness of the photodynamic therapy done in BB rats was demonstrated, may correspond to a dose of 0.5 mg/kg to 1.0 mg/kg of the drug in a human, on the basis of comparison between human and rat in respect of the change of the concentration of the drug in serum.

### TEST EXAMPLE 4

A sodium salt of the mono-L-aspartyl chlorin e6-gold complex was administered at a dose of 5 mg/kg via a tail vein in the same manner as in Test Example 3 into BB rats whose urine sugar became positive. Twenty-four hours after the administration, a coherent X-ray of a wavelength of 0.153 Å, which is specific to the absorption of electrons in the K shell of the gold atoms in the mono-L-aspartyl chlorin e6-gold complex, was applied (at Spring-8 radiation facility, at energy of 25 KeV) transcutaneously from the outside via the back of the rats to the target sites, that is, the Langerhans islets in the pancreas and their surrounding region. The X-ray having passed through the body of rat was photographed on an X-ray film (Fuji IX25) and its X-ray absorption image was observed, revealing that the mono-L-aspartyl chlorin e6-gold complex was accumulated in the Langerhans islets in the pancreas.

Further, the survival rate (%) of the rats as treated by the photodynamic therapy using the mono-L-aspartyl chlorin e6-gold complex was evaluated in comparison with that of the control group of rats (untreated).

As a result, it was recognized that, like Test Example 3, the survival curve of the group of rats treated by the photodynamic therapy was evidently prolonged or improved as compared with the control group of rats.

The dose of 5 mg/kg of the mono-L-aspartyl chlorin e6-gold complex, with which the efficacy of the photodynamic therapy done in BB rats was demonstrated, may correspond to a dose of about 0.5 mg/kg to 1.0 mg/kg of said gold complex in a human, on the basis of comparison between human and rat in respect of the change of the concentration of the drug in serum.

What is claimed is:

1. A method for diagnosing insulin-dependent diabetes mellitus, which comprises:
administering to a mammal a sufficient amount of a photosensitizer for accumulating the photosensitizer in the macrophages having infiltrated the inflammatory sites of Langerhans islets in a pancreas of the mammal, the photosensitizer being capable of accumulating specifically in the macrophages having infiltrated the inflammatory sites of Langerhans islets in the pancreas of the mammal and being activated upon absorption of a light ray of a specific wavelength;
irradiating the pancreas with the light ray of the specific wavelength; and
detecting fluorescence of the photo-excited photosensitizer in the inflammatory sites of Langerhans islets.

2. The diagnostic method according to claim 1, wherein the photosensitizer is a member selected from the group consisting of chlorin e6, a chlorin e6 derivative, a benzoporphyrin derivative, rostaporfin, motexafin lutetium, ATX-S10, temoporfin and porfimer sodium, and a pharmaceutically acceptable salt thereof.

3. The diagnostic method according to claim 1, wherein the photosensitizer is talaporfin, namely mono-L-aspartyl chlorin e6 represented by formula (I): 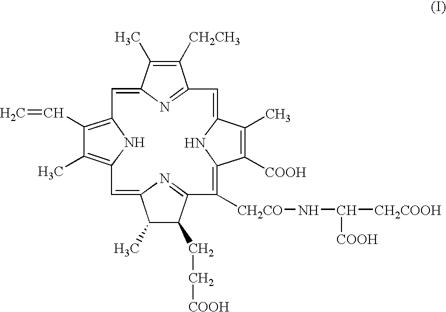
or a pharmaceutically acceptable salt or a solvate thereof.

4. A method for diagnosing insulin-dependent diabetes mellitus, which comprises:
administering to a mammal a sufficient amount of an X-ray absorbing substance for accumulating the X-ray absorbing substance in the macrophages having infiltrated the inflammatory sites of Langerhans islets in a pancreas of the mammal, the X-ray absorbing substance being capable of accumulating specifically in the macrophages having infiltrated the inflammatory sites of Langerhans islets in the pancreas and being activated upon absorption of an X-ray of a specific wavelength;
irradiating the pancreas with the X-ray of the specific wavelength; and
detecting an X-ray absorption image in the inflammatory sites of Langerhans islets.

5. The diagnostic method according to claim 4, wherein the X-ray absorbing substance is a mono-L-aspartyl chlorin e6-gold complex represented by formula (II): 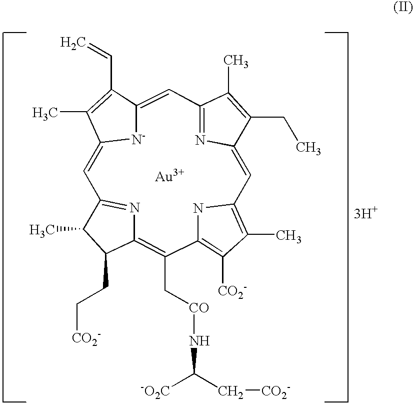
or a pharmaceutically acceptable salt or a solvate thereof.

6. The diagnostic method according to claim 5, wherein the X-rays as irradiated are X-rays containing an X-ray of 0.153 Å wavelength.

7. A method for treating therapeutically insulin-dependent diabetes mellitus, which comprises:
administering to a mammal with the diabetes mellitus a sufficient amount of a photosensitizer for accumulating the photosensitizer in the macrophages having infiltrated the inflammatory sites of Langerhans islets in a pancreas of the mammal, the photosensitizer being capable of accumulating specifically in the macrophages having infiltrated the inflammatory sites of Langerhans islets in the pancreas and being capable of generating, upon absorption of a light ray of a specific wavelength, active oxygen for damaging or destroying the macrophages; and
irradiating the inflammatory sites of Langerhans islets with the light ray of the specific wavelength at an irradiance and for a time sufficient to damage or destroy the macrophages having infiltrated the Langerhans islets.

8. The method according to claim 7, wherein the photosensitizer is a member selected from the group consisting of chlorine e6, a chlorin e6 derivative, a benzoporphyrin derivative, rostaporfin, motexafin lutetium, ATX-S10, temoporfin and porfimer sodium, and a pharmaceutically acceptable salt thereof.

9. The method according to claim 7, wherein the photosensitizer is talaporfin, namely mono-L-aspartyl chlorin e6 represented by formula (I): 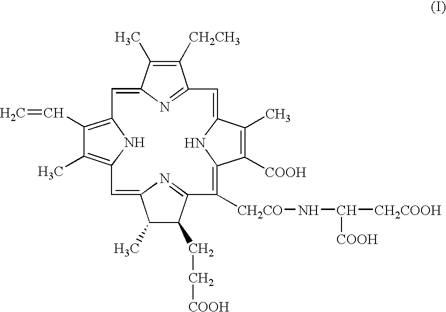
or a pharmaceutically acceptable salt or a solvate thereof.

10. A method for treating therapeutically insulin-dependent diabetes mellitus, which comprises:
administering to a mammal with the diabetes mellitus a sufficient amount of an X-ray absorbing substance for accumulating the X-ray absorbing substance in the macrophages having infiltrated the inflammatory sites of Langerhans islets in a pancreas of the mammal, the X-ray absorbing substance being capable of accumulating specifically in the macrophages having infiltrated the inflammatory sites of Langerhans islets in the pancreas and being activated upon absorption of an X-ray of a specific wavelength; and
irradiating the pancreas with the X-ray of the specific wavelength by irradiation of a beam of X-rays at an intensity and for a time sufficient to damage or destroy the macrophages having infiltrated the Langerhans islets.

11. The method according to claim 10, wherein the X-ray absorbing substance is a mono-L-aspartyl chlorin e6-gold complex represented by formula (II): 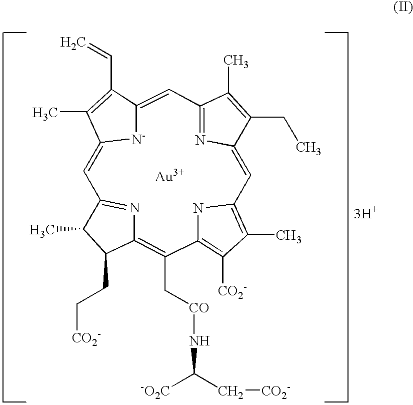
or a pharmaceutically acceptable salt or a solvate thereof.

12. The method according to claim 10, wherein the X-rays as irradiated are X-rays containing an X-ray of 0.153 Å wavelength.
